(19) United States
(12) Patent Application Publication
(10) Pub. No.: US 2011/0183398 A1
(43) Pub. Date: Jul. 28, 2011

(54) MICROORGANISM-CAPTURING COMPOSITIONS AND METHODS

(75) Inventors: Sridhar V. Dasaratha, Bangalore (IN); G.M. Bommarito, Stillwater, MN (US); Tushar A. Kshirsagar, Woodbury, MN (US)

(73) Assignee: 3M INNOVATIVE PROPERTIES, St. Paul, MN (US)

(21) Appl. No.: 12/811,142

(22) PCT Filed: Dec. 23, 2008
(86) PCT No.: PCT/US2008/088099

§ 371 Date: Jun. 29, 2010

Related U.S. Application Data
(60) Provisional application No. 61/018,011, filed on Dec. 31, 2007.

(51) Int. Cl. C12N 11/10; C12N 1/20; C12M 1/00

(52) U.S. Cl. 435/178; 435/252.1; 435/287.1

(57) ABSTRACT

The invention relates to compositions, methods, devices, and kits for non-specifically isolating bacterial cells. The compositions comprise a solid support which has a surface comprising a combination of a carbohydrate and a biotin-binding protein, wherein the protein is covalently bonded to the carbohydrate, and wherein the protein is linked to the solid support via the carbohydrate; and at least one of 1) a plurality of bacterial cells non-specifically bound to the combination of the carbohydrate and the protein or 2) an amphiphilic glycoside of a steroid or triterpene. The methods, devices, and kits include at least one of these compositions.

### CROSS REFERENCE TO RELATED APPLICATIONS

The present invention claims priority to U.S. Provisional Application Ser. No. 61/018,011, filed Dec. 31, 2007, which is incorporated herein by reference.

### GOVERNMENT RIGHTS

The U.S. Government may have certain rights to this invention under the terms of Contract Nos. DAAD-13-03-C-0047 (Program No. 2640) and W81XWH-07-01-0354 (Program No. 2750) granted by the Department of Defense.

### BACKGROUND

Assays for determining the presence of microorganisms in a variety of samples, including food, clinical, environmental, and experimental samples, are of increasing importance. Such assays can provide an indication of microorganism load and/or identification of microorganisms that are present.

Current techniques for qualitatively and quantitatively determining the presence of microorganisms typically involve identification of a specific microorganism, such as a pathogen. Successful detection of the presence of a particular microorganism, such as a bacterium, in a sample depends upon the concentration of the bacterium in the sample. Generally, bacterial samples are cultured to assess viability and increase the number of bacteria in the sample to assure an adequate level for detection. The culturing step requires substantial time, which delays obtaining the detection results.

By concentrating bacteria in a sample, detection can be carried out using a shorter or even no culturing step. Methods have been developed to isolate, and thereby concentrate, specific bacterial strains by using antibodies specific to the strain.

Other concentration methods that are non-strain specific, which would allow a more general sampling of the microorganisms present, have been proposed. Once isolated, a specific strain or specific strains within a mixture of strains can be identified and/or quantified using known detection methods, including, for example, nucleic acid amplification methods.

Non-specific isolation of microorganisms using carbohydrate and lectin protein interactions has been proposed. Lectins present on the surface of bacteria were suggested as capture targets for certain carbohydrates attached to polyacrylamide. Substances that serve as nutrients for microorganisms have also been proposed for use as ligands for non-specific capture of microorganisms. Such nutrient ligands included carbohydrates, vitamins, iron-chelating compounds, and sidorophores. Supported chitsan has also been proposed for concentrating microorganisms in a non-strain-specific manner, allowing the microorganisms to be more easily and rapidly assayed.

Although some methods for isolating microorganisms have been described, there continues to be an interest in and a need for improved materials and methods for isolating microorganisms.

### SUMMARY

It has now been found that the combination of a biotin-binding protein attached to a solid support via a carbohydrate is effective for non-specifically binding bacterial cells. Compositions with a carbohydrate on a solid support, but without the biotin-binding protein, were found to be less effective for non-specifically binding bacterial cells. Moreover, compositions with a biotin-binding protein on the solid support, but without the carbohydrate, were also found to be less effective for non-specifically binding bacterial cells. In some embodiments, the biotin-binding protein attached to the solid support via the carbohydrate is even more effective for non-specifically binding bacterial cells in the presence of an amphiphilic glycoside of a steroid or triterpene.

The present invention, therefore, provides new compositions for non-specifically binding bacterial cells.

In one embodiment, there is provided a composition comprise a solid support which has a surface comprising a combination of a carbohydrate and a biotin-binding protein, wherein the protein is covalently bonded to the carbohydrate, and wherein the protein is linked to the solid support via the carbohydrate; and at least one of 1) a plurality of bacterial cells non-specifically bound to the combination of the carbohydrate and the protein or 2) an amphiphilic glycoside of a steroid or triterpene.

In another embodiment, there is provided a composition comprising:

a solid support which has a surface comprising a combination of a carbohydrate and a biotin-binding protein, wherein the protein is covalently bonded to the carbohydrate, and wherein the protein is linked to the solid support via the carbohydrate; and

a plurality of bacterial cells non-specifically bound to the combination of the carbohydrate and the protein.

In another embodiment, there is provided a composition comprising:

a solid support which has a surface comprising a combination of a carbohydrate and a biotin-binding protein, wherein the protein is covalently bonded to the carbohydrate, and wherein the protein is linked to the solid support via the carbohydrate; and

an amphiphilic glycoside of a steroid or triterpene.

In another aspect, there is provided a method for isolating bacterial cells comprising:

providing a solid support which has a surface comprising a combination of a carbohydrate and a biotin-binding protein, wherein the protein is covalently bonded to the carbohydrate, and wherein the protein is linked to the solid support via the carbohydrate;

providing a sample suspected of having a plurality of bacterial cells;

contacting the solid support which has the surface comprising the combination of the carbohydrate and the biotin-binding protein with the sample; wherein at least a portion of the plurality of bacterial cells from the sample become non-specifically bound to the surface of the solid support; and

separating the solid support from the remainder of the sample after the at least a portion of the plurality of bacterial cells from the sample become non-specifically bound to the surface of the solid support.

In another aspect, there is provided a device for detecting bacterial cells comprising:

a composition comprising:
a solid support which has a surface comprising a combination of a carbohydrate and a biotin-binding protein, wherein the protein is covalently bonded to the carbohydrate, and wherein the protein is linked to the solid support via the carbohydrate; and
a plurality of bacterial cells non-specifically bound to the combination of the carbohydrate and the protein; and

a means for detecting the bacterial cells.

In another embodiment, there is provided a device for detecting bacterial cells comprising:

a composition comprising:
a solid support which has a surface comprising a combination of a carbohydrate and a biotin-binding protein, wherein the protein is covalently bonded to the carbohydrate, and wherein the protein is linked to the solid support via the carbohydrate; and
an amphiphilic glycoside of a steroid or triterpene; and

a means for detecting the bacterial cells.

In another aspect, there is provided a kit comprising:
a solid support which has a surface comprising a combination of a carbohydrate and a biotin-binding protein, wherein the protein is covalently bonded to the carbohydrate, and wherein the protein is linked to the solid support via the carbohydrate; and
an amphiphilic glycoside of a steroid or triterpene.

### DEFINITIONS

The term “amphiphilic glycoside of a steroid or triterpene” refers to a steroid or triterpene joined to a sugar by a glycosidic linkage, the resulting material having amphiphilic properties, that is, surfactant properties, the material being both hydrophilic and lipophilic.

The term “biotin-binding protein” refers to a protein which has a high affinity and selectivity for binding biotin. As used herein, the “biotin-binding protein” does not include bound biotin.

The term “magnetic particles” means particles, particle conglomerates, or beads comprised of ferromagnetic, paramagnetic, or superparamagnetic particles, including dispersions of said particles in a polymer bead.

As used herein, “a”, “an”, “the”, “at least one”, and “one or more” are used interchangeably.

The terms “comprises” and variations thereof do not have a limiting meaning where these terms appear in the description and claims.

The above summary of the present invention is not intended to describe each disclosed embodiment or every implementation of the present invention. The description that follows more particularly exemplifies illustrative embodiments. In several places throughout the description, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS OF THE INVENTION

The present invention provides new compositions for non-specifically binding bacterial cells. “Non-specifically binding” bacterial cells means that the binding is not specific to any type of bacterial cells. Thus, all bacteria in a sample can be isolated from other components in the sample rather than targeting, for example, one strain of bacteria. Both gram positive and gram negative bacteria can be bound. The resulting isolated bacteria can then be subjected to known detection methods, such as bacterial load detection.

In one embodiment, there is provided a composition comprising: a solid support which has a surface comprising a combination of a carbohydrate and a biotin-binding protein, wherein the protein is covalently bonded to the carbohydrate, and wherein the protein is linked to the solid support via the carbohydrate; and a plurality of bacterial cells non-specifically bound to the combination of the carbohydrate and the protein.

In another embodiment, there is provided a composition comprising: a solid support which has a surface comprising a combination of a carbohydrate and a biotin-binding protein, wherein the protein is covalently bonded to the carbohydrate, and wherein the protein is linked to the solid support via the carbohydrate; and an amphiphilic glycoside of a steroid or triterpene. For certain embodiments, this composition further comprises a plurality of bacterial cells non-specifically bound to the combination of the carbohydrate and the biotin-binding protein.

The compositions can be prepared by combining the solid support having the combination of carbohydrate and biotin-binding protein described above with at least one of a plurality of bacterial cells or an amphiphilic glycoside of a steroid or triterpene. The solid support having the combination of carbohydrate and biotin-bind protein can be provided as described below. Combining the solid support with the bacterial cells and/or amphiphilic glycoside can be conveniently carried out by suspending or immersing the solid support in a buffer and adding the bacterial cells, also suspended in a buffer, and/or the amphiphilic glycoside dissolved in a buffer. The amphiphilic glycoside buffer solution preferably contains about 0.2 to about 10 weight/volume percent (% w/v) amphiphilic glycoside, more preferably about 0.5 to about 5% w/v.

In another embodiment, there is provided a method for isolating bacterial cells comprising: providing a solid support which has a surface comprising a combination of a carbohydrate and a biotin-binding protein, wherein the protein is covalently bonded to the carbohydrate, and wherein the protein is linked to the solid support via the carbohydrate; providing a sample suspected of having a plurality of bacterial cells; contacting the solid support which has the surface comprising the combination of the carbohydrate and the biotin-binding protein with the sample; wherein at least a portion of the plurality of bacterial cells from the sample become non-specifically bound to the surface of the solid support; and separating the solid support from the remainder of the sample after the at least a portion of the plurality of bacterial cells from the sample become non-specifically bound to the surface of the solid support.

The sample suspected of having a plurality of bacterial cells can be provided from a wide range of sources using known methods. Examples of sources include physiological fluid, e.g., blood, saliva, ocular lens fluid, synovial fluid, cerebral spinal fluid, pus, sweat, exudate, urine, mucus, mucosal tissue (e.g., buccal, gingival, nasal, ocular, tracheal, bronchial, gastrointestinal, rectal, urethral, ureteral, vaginal, cervical, and uterine mucosal membranes), lactation milk, or the like. Further examples of sample sources include those obtained from a body site, e.g., wound, skin, nares, nasopharyngeal cavity, nasal cavities, anterior nasal vestibule, scalp, nails, outer ear, middle ear, mouth, rectum, or other site. Additional examples of sample sources include process streams, water, food, soil, vegetation, air (e.g., contaminated), surfaces, such as food preparation surfaces, and the like.

Known sampling techniques can be used to gather the sample from the source. For example, a volume of a liquid sample can be drawn from the source, or a swab or wipe can be used to collect a sample from a physiological or environmental surface. A wide variety of swabs or other sample collection devices are commercially available.

The sample can be provided for use in the above method directly as is from the collection device, for example, in the case of aqueous liquids. Alternatively, the sample can be provided by eluting, releasing, or washing the sample from the collection device using, for example, water, physiological saline, pH buffered solutions, or any other solutions or combinations of solutions that can remove the sample from the collection device and bring the sample into suspension. An example of an eluting buffer is phosphate buffered saline (PBS), which can be used in combination with a surfactant, such as polyoxyethylene sorbitan monolaurate or a poly(oxyethylene-co-oxypropylene) block copolymer. Other extraction solutions can function to maintain specimen stability during transport from a sample collection site to a sample analysis site. Examples of these types of extraction solutions include Amies' and Stuart's transport media.

The sample may be subjected to a treatment prior to contact with the solid support, such as dilution of viscous fluids, concentration, filtration, distillation, dialysis, dilution, inactivation of natural components, addition of reagents, chemical treatment, or the like.

Contacting the solid support which has the surface comprising the combination of the carbohydrate and the biotin-binding protein with the sample suspected of having a plurality of bacterial cells can be conveniently carried out by suspending or immersing the solid support in a buffer and adding the sample, which may also be suspended in a buffer. Suitable buffers include PBS and PBS with a nonionic surfactant. The mixture can be agitated for a period of time sufficient to allow the bacterial cells to become bound to the combination of carbohydrate and biotin-binding protein on the solid support. The binding can be conveniently carried out at room temperature.

Separating the solid support from the remainder of the sample can be carried out using well known methods. For example, the remainder of the sample can be drained, decanted, drawn, centrifuged, pipetted, and/or filtered off of the solid support. When the solid support is magnetic particles the solid support can be consolidated in one area of the container with a magnet for convenient removal of the remainder of the sample, for example, by decanting, pipetting, or forcing the supernate out of the container using a pressure differential or a g-force. Additionally, the solid support may be washed, for example, with a buffer, to remove any remaining unbound sample material.

For certain embodiments, the method for isolating bacterial cells further comprises detecting the at least a portion of the plurality of bacterial cells. For certain of these embodiments, the detecting is carried out by a detection method selected from the group consisting of adenosine triphosphate (ATP) detection by bioluminescence, polydiacetylene (PDA) colorimetric detection, nucleic acid detection, immunological detection, growth based detection, surface acoustic wave detection, or the like.

ATP detection can be used as a nonspecific indicator of bacterial load. After separating the solid support with non-specifically bound bacterial cells from the remainder of the sample (which may contain interfering components such as extra-cellular ATP), the cells are lysed and contacted with luciferin and luciferase. The resulting bioluminescence, which is of an intensity proportional to the number of captured bacterial cells, is then measured, for example, using a luminometer.

PDA colorimetric detection can be used to detect specific bacteria or a spectrum of bacteria by contacting a colorimetric sensor with the bacteria. The colorimetric sensor comprises a receptor and a polymerized composition which includes a diacetylene compound or a polydiacetylene. When bacterial cells are bound by the receptor, resulting conformational changes to the sensor cause a measurable color change. The color change can be measured, for example, visually or using a colorimeter. Indirect detection of bacterial cells using probes which can bind to the receptor may also be used. PDA colorimetric detection using such colorimetric sensors is known and described, for example, in U.S. Patent Application Publication No. 2006/0134796A1, International Publication Nos. WO 2004/057331A1 and WO 2007/016633A1, and in Assignee's co-pending U.S. Patent Application Ser. No. 60/989,298.

Methods for detecting nucleic acids, including DNA and RNA, often include amplifying or hybridizing the nucleic acids. The captured bacterial cells are lysed to make the cellular nucleic acids available for detection. Lysing can be carried out enzymatically, chemically, and/or mechanically. Enzymes used for lysis include, for example, lysostaphin, lysozyme, mutanolysin, or others. Chemical lysis can be carried out using a surfactant, alkali, heat, or other means. When alkali is used for lysis, a neutralization reagent may be used to neutralize the solution or mixture after lysis. Mechanical lysis can be accomplished by mixing or shearing using solid particles or microparticles such as beads or microbeads. Sonication may also be used for lysis. The lysis reagent can include a surfactant or detergent such as sodium dodecylsulfate (SDS), lithium laurylsulfate (LLS), TRITON series, TWEEN series, BRIJ series, NP series, CHAPS, N-methyl-N-(1-oxododecyl)glycine, sodium salt, or the like, buffered as needed; a chaotrope such as guanidium hydrochloride, guanidium thiacyanate, sodium iodide, or the like; a lysis enzyme such as lysozyme, lysostaphin, mutanolysin, proteinases, pronases, cellulases, or any of the other commercially available lysis enzymes; an alkaline lysis reagent; solid particles such as beads, or a combination thereof.

Examples of amplification methods include polymerase chain reaction (PCR); target polynucleotide amplification methods such as self-sustained sequence replication (3SR) and strand-displacement amplification (SDA); methods based on amplification of a signal attached to the target polynucleotide, such as “branched chain” DNA amplification; methods based on amplification of probe DNA, such as ligase chain reaction (LCR) and QB replicase amplification (QBR); transcription-based methods, such as ligation activated transcription (LAT), nucleic acid sequence-based amplification (NASBA), amplification under the trade name INVADER, and transcription-mediated amplification (TMA); and various other amplification methods, such as repair chain reaction (RCR) and cycling probe reaction (CPR).

Primer directed nucleic acid amplification methods, which include, for example, thermal cycling methods such as PCR, LCR, and SDA, may be used advantageously for detecting a spectrum of bacteria by choosing a primer which can hybridize to nucleic acids from the spectrum of bacteria.

Nucleic acid hybridization detection methods are also well known. Here, a single stranded nucleic acid probe is hybridized to a single stranded nucleic acid(s) from the bacterial cells to provide a double stranded nucleic acid which includes the probe strand. Nucleic acid probes (probe labels) such as fluorescent, chemiluminescent, and radioactive labels which can then be quantified and detected are known. Moreover, a species specific probe or a combination of species specific probes may be used to detect a specific bacteria or a number of different bacteria.

Immunological detection includes detection of a biological molecule, such as a protein, proteoglycan, or other material with antigenic activity, acting as a marker on the surface of bacteria. Detection of the antigenic material is typically by an antibody, a polypeptide selected from a process such as phage display, or an aptamer from a screening process. Immunological detection methods are known, examples of which include immunoprecipitation and enzyme-linked immunosorbent assays (ELISA). Antibody binding can be detected in several ways, including by labeling either the primary or the secondary antibody with a fluorescent dye, quantum dot, or an enzyme that can produce chemiluminescence or a color change. Plate readers and lateral flow devices have been used for detecting and quantifiying the binding event.

Growth based detection methods are well known and generally include plating the bacteria, culturing the bacteria to increase the number of bacterial cells under specific conditions, and enumerating the bacterial cells. PETRIFILM Aerobic Count Plates (3M Company, St. Paul, Minn.) can be used for this purpose.

Surface acoustic wave detection, described, for example, in International Publication No. WO 2005/071416, is also known for detecting bacterial cells. For example, a bulk acoustic wave-impedance sensor has been used for detecting the growth and numbers of bacterial cells on the surface of a solid medium. The concentration range of the bacteria that can be detected by this method was 3.4×10² to 6.7×10⁶ cells/ml. See Le Deng et al., J. Microbiological Methods, Vol. 26, Iss. 10-2, 197-203 (1997).

For certain embodiments, including any one of the above embodiments of the method for isolating bacterial cells, contacting the solid support with the sample is carried out in the presence of an amphiphilic glycoside of a steriod or triterpene.

As indicated above, contacting the solid support with the sample may be carried out also in the presence of a buffer, which may provide a suitable media for effectively binding the bacterial cells to the solid support. A buffer of appropriate charge, osmolarity, or other characteristic may be added to the sample prior to, simultaneously with, or after contact with the solid support. PBS and PBS-L64 buffers are examples of such cell binding buffers.

In another embodiment, there is provided a device for detecting bacterial cells comprising: a composition comprising a solid support which has a surface comprising a combination of a carbohydrate and a biotin-binding protein, wherein the protein is covalently bonded to the carbohydrate, and wherein the protein is linked to the solid support via the carbohydrate, and a plurality of bacterial cells non-specifically bound to the combination of the carbohydrate and the protein; and a means for detecting the bacterial cells.

In another embodiment, there is provided a device for detecting bacterial cells comprising: a composition comprising a solid support which has a surface comprising a combination of a carbohydrate and a biotin-binding protein, wherein the protein is covalently bonded to the carbohydrate, and wherein the protein is linked to the solid support via the carbohydrate, and an amphiphilic glycoside of a steroid or triterpene; and a means for detecting the bacterial cells. For certain embodiments, the composition further comprises a plurality of bacterial cells non-specifically bound to the combination of the carbohydrate and the protein.

The device may include one or more structural features which contain the composition and the means for detecting the bacterial cells. The device can provide a location or locations and conditions for capturing bacterial cells by the solid support, separating the remaining sample from the solid support, and detecting the bacterial cells. The sample may be located in one or a plurality of locations or reservoirs. The device may provide uniform and accurate temperature control of one or more of the locations or reservoirs. The device may provide channels between locations or reservoirs, for example, such that bacterial cell binding may take place in one or more locations or reservoirs, and bacterial cell detection may take place in one or more other locations or reservoirs. For certain embodiments, including any one of the above embodiments which include the device for detecting bacterial cells, the device is a lateral flow device, a vertical flow device, or a combination thereof. Some examples of such devices are described in Assignee's co-pending U.S. Patent Application Ser. No. 60/989,291. For certain embodiments, the device is a microfluidic device. Some examples of microfluidic devices are described in U.S. Publication Numbers 2002/0064885 (Bedingham et al.); US2002/0048533 (Bedingham et al.); US2002/0047003 (Bedingham et al.); and US2003/138779 (Parthasarathy et al.); U.S. Pat. Nos. 6,627,159; 6,720,187; 6,734,401; 6,814,935; 6,987,253; 7,026,168, and 7,164,107; and International Publication No. WO 2005/061084 A1 (Bedingham et al.).

For certain embodiments, including any one of the above embodiments of a composition, a method, or a device, which includes a plurality of bacterial cells, the plurality of bacterial cells includes two or more different types of bacteria. A type of bacteria or a type of bacterial cells refers to a strain, species, genus, family, order, or Part of the bacteria or bacterial cells.

For certain embodiments, including any one of the above embodiments of a composition, a method, or a device, which includes a plurality of bacterial cells, the bacteria are selected from the group consisting of gram positive bacteria and gram negative bacteria. For certain of these embodiments, the bacteria are selected from the group consisting of Bacillus, Bordetella, Borrelia, Campylobacter, Clostridium, Corynebacteria, Enterobacter, Enterococcus, Escherichia, Helicobacter, Legionella, Listeria, Mycobacterium, Neisseria, Pseudomonas, Salmonella, Shigella, Staphylococcus, Streptococcus, Vibrio, and Yersinia. For certain embodiments, the bacteria are selected from the group consisting of S. aureus, P. aeruginosa, S. epidermidis, E. faecalis, Strep agalatiae, Strep dysgalatiae, E. coli, Salmonella, and Group B Strep.

In another embodiment, there is provided a kit comprising a solid support which has a surface comprising a combination of a carbohydrate and a biotin-binding protein, wherein the protein is covalently bonded to the carbohydrate, and wherein the protein is linked to the solid support via the carbohydrate; and an amphiphilic glycoside of a steroid or triterpene. For certain of these embodiments, the kit further comprises a means for detecting bacterial cells.

For certain embodiments, including any one of the above embodiments of a device or kit, the means for detecting the bacterial cells is selected from the group consisting of reagents for detecting adenosine triphosphate (ATP) by bioluminescence, a PDA colorimetric sensor, reagents for nucleic acid detection, reagents for immunological detection, media for plating and enumerating bacterial cells, a surface acoustic wave sensor, and the like.

Reagents for detecting ATP include luciferin, luciferase, and optionally a lysing agent. Reagents for nucleic acid detection include, for example a primer, a probe, an enzyme for extending the primer, or a combination thereof. Reagents for immunological detection include, for example, at least one antibody.

For certain embodiments, including any one of the above embodiments of a composition, a method, a device, or a kit, which includes an amphiphilic glycoside of a steroid or triterpene, the amphiphilic glycoside of the steroid or triterpene is a saponin. Saponins are 27 carbon atom steroids or 30 carbon atom triterpenes joined to a sugar by a glycosidic linkage. For certain embodiments, the sugar is selected from the group consisting of hexoses, pentoses, saccharic acids, and a combination thereof. Saponin has been used as a mild detergent and for lysing red blood cells. However, unexpectedly it has now been found that this class of materials can be present with the combination of the biotin-binding protein and the carbohydrate attached to the solid support via the carbohydrate without loss of effectiveness for non-specifically binding bacterial cells. Moreover, for certain embodiments, the combination of the biotin-binding protein and the carbohydrate attached to the solid support via the carbohydrate, in combination with the amphiphilic glycoside of a steroid or triterpene, or an embodiment thereof described above, is even more effective for non-specifically binding bacterial cells.

For certain embodiments, including any one of the above embodiments of a composition, a method, a device, or a kit, the biotin-binding protein is a protein which binds four biotin molecules per protein molecule if biotin is present with the protein. Although the embodiments of the present invention include a biotin-binding protein, the biotin-binding protein is included without biotin (or biotin attached to another material) bound to the biotin-binding protein. For certain of these embodiments, the biotin-binding protein is selected from the group consisting of avidin, streptavidin, neutravidin, and selectively nitrated avidin. Avidin is a glycoprotein with a mass of about 66 kDa and an isoelectric point of 10 to 10.5. About 10 percent of avidin's total mass is carbohydrate. Avidin is commercially available, for example, from Sigma-Aldrich. Streptavidin is a tetrameric protein with a mass of about 60 kDa and an isoelectric point of about 5. Streptavidin, which lacks the carbohydrate component found in avidin, is commercially available, for example, from Pierce. Neutravidin is a deglycosylated avidin, with a mass of about 60 kDa and an isoelectric point of about 6.3. Neutravidin is commercially available from Pierce. Selectively nitrated avidin, available from Molecular Probes, Inc. under the trade name CAPTAVIDIN, has tyrosine residues in the four biotin-binding sites of avidin nitrated. For certain of these embodiments, the biotin-binding protein is streptavidin.

For certain embodiments, including any one of the above embodiments of a composition, a method, a device, or a kit, the carbohydrate is selected from the group consisting of monsaccharides, oligosaccharides, polysaccharides, and combinations thereof. Suitable monosaccharides include, for example, mannose, galactose, glucose, fructose, fucose, N-acetylglucosamine, N-acetylgalactosamine, rhamnose, galactosamine, glucosamine, galacturonic acid, glucuronic acid, N-acetylneuraminic acid, methyl D-mannopyranoside, α-methylglucoside, galactoside, ribose, xylose, arabinose, saccharate, mannitol, sorbitol, inositol, glycerol, a derivative of any one of these monosaccharides, and a combination thereof. Suitable oligosaccharides include those having 2 to 12 monosaccharide units, which may be the same or different. Examples include oligomannose having 2 to 6 units, maltose, sucrose, trehalose, cellobiose, salicin, a derivative of any one of these oligosaccharides, and a combination thereof. Suitable polysaccharides include those having more than 12 monosaccharide units, which may be the same or different. Examples include polysaccharides such as gum arabic (acacia gum), believed to be a branched polymer of galactose, rhamnose, arabinose, and glucuronic acid as the calcium, magnesium, and potassium salts; galactomannan polysaccharide (locust bean gum), believed to be a straight chain polymer of mannose and one galactose branch on every fourth mannose; guar gum, believed to be a straight chain polymer of mannose and one galactose branch on every other unit; and gum karaya, believed to include a partially acetylated polymer of galactose, rhamnose, and glucuronic acid. For certain of these embodiments, the carbohydrate includes at least one carboxy group. Any one of the above described monsaccharides, oligosaccharides, polysaccharides, and a combination thereof, which includes at least one carboxy group, may be used. Moreover, any one of the above described monsaccharides, oligosaccharides, polysaccharides, and a combination thereof, which is derivatized to include at least one carboxy group, may be used. For certain of these embodiments, the carbohydrate is a polysaccharide. For certain of these embodiments, the carbohydrate comprises arabic acid.

The biotin-binding protein can be covalently bonded to the carbohydrate by reaction(s) between functional groups on the biotin-binding protein and functional groups on the carbohydrate. For certain embodiments, including any one of the above embodiments of a composition, a method, a device, or a kit, the carbohydrate includes at least one carboxy group, and the biotin-binding protein is covalently bonded to the carbohydrate through a linking group, the linking group being the reaction product of the protein and the at least one carboxy group of the carbohydrate. The biotin-binding protein becomes covalently bonded to the carbohydrate by well known interactions. For example, an amino group of the biotin-binding protein reacts with a carboxy group of the carbohydrate to provide the linking group, in this case, an amido group. In another example, a hydroxy group of the biotin-binding protein reacts with a carboxy group of the carbohydrate to provide the linking group, in this case, an ester group. Either or both of these linking groups can provide the bond between the protein and the carbohydrate, although other known bonding routes and linking groups may be used additionally or alternatively.

The solid support may be comprised partially or completely of the carbohydrate. The solid support is structured with a carbohydrate at a surface of the solid support, such that the carbohydrate is available for reacting with a biotin-binding protein. The biotin-binding protein is then bonded to the carbohydrate, which in turn is attached to the solid support or is the solid support.

The solid support may be any of the known supports or matrices which are currently used for separation or immobilization. For certain embodiments, including any one of the above embodiments of a composition, a method, a device, or a kit, the solid support is selected from the group consisting of a bead, a gel, a film, a sheet, a particle, a filter, a membrane, a plate, a strip, a tube, a well, a fiber, a capillary, and a combination thereof. The solid support may comprise a glass, silica, ceramic, metal, polymer, or combination thereof. Suitable polymers include, for example, latex, cellulose, polysaccharides, polyacrylamide, polymethacrylates, polyolefins, such as polyethylene, polypropylene, and poly(4-methylbutene), polyolefin copolymers, polyolefin ionomers, polyolefin blends, polystyrene, polyamides, such as a nylon, poly(vinylbutyrate), polyesters, such as poly(ethyleneterephthalate), polyvinylchloride, poly(vinyl alcohol), and polycarbonate. For certain embodiments, the solid support comprises a polysaccharide.

The solid support may be coated with a carbohydrate such as one or more of those described above. The coated carbohydrate may be bound to the solid support by known bonding methods and structures. For example, the solid support surface may first be functionalized with isocyanate groups and then coated with the carbohydrate. Hydroxy groups on the carbohydrate (and amino groups, if present) react with the isocyanate groups, bonding the carbohydrate to the solid support.

For certain embodiments, including any one of the above embodiments of a composition, a method, a device, or a kit, the solid support is magnetic particles. A variety of magnetic particles are commercially available, including, for example, polymer particles based on poly(vinyl alcohol) in which a magnetic colloid has been encapsulated (Chemagen AG, Germany), polystyrene spheres including a dispersion of a mixture of maghemite (gamma-Fe₂O₃) and magnetite (Fe₃O₄), and a polystyrene shell (Dynal Biotech ASA, Oslo, Norway), magnetic particles with a polysaccharide matrix (Chemicell GmbH, Berlin, Germany), and magnetic particles with a polysaccharide core and coated with streptavidin (Chemicell GmbH). Those magnetic beads or particles which do not include a carbohydrate available for bonding with a biotin-binding protein may be modified as, for example, described above to attach a carbohydrate, to which a biotin-binding protein can then be bonded. The magnetic particles with the polysaccharide core or matrix can be reacted with a biotin-binding protein to provide the above described combination of carbohydrate and protein. The magnetic particles with the polysaccharide core or matrix already coated with streptavidin may be used without modification.

For certain embodiments, including any one of the above embodiments which includes magnetic beads or particles, the magnetic beads or particles have a diameter of about 0.02 to about 5 microns. For beads or particles which are not spherical, this diameter refers to at least one dimension of the bead or particle. This diameter range provides a suitable surface area for effective non-specific capture of bacterial cells.

Objects and advantages of this invention are further illustrated by the following examples, but the particular materials and amounts thereof recited in these examples, as well as other conditions and details, should not be construed to unduly limit this invention.

### EXAMPLES

All parts, percentages, ratios, etc. in the Examples and throughtout the specification are by mole unless indicated otherwise. All solvents and reagents without a named supplier were purchased from Aldrich Chemical; Milwaukee, Wis. Water was purified by the use of a U-V Milli-Q water purifier with a resistivity of 18.2 Mohms/cm (Millipore, Bedford Mass.).

Table of Abbreviations

| Abbreviation or | |
|---|---|
| Trade Name | Description |
| ATCC | American Type Culture Collection |
| PBS buffer | A phosphate buffer saline (PBS) solution |
| | prepared by diluting ten-fold a 10x PBS |
| | liquid concentrate available from EMD |
| | Biosciences, San Diego CA |
| PBS L64 buffer | prepared by taking the PBS buffer solution |
| | and adding 0.2% (w/v) of PLURONIC L64 |
| PLURONIC L64 | Trade designation for a hydroxy terminated |
| | poly(oxyethylene)poly(oxypropylene)poly(oxy- |
| | ethylene) block copolymer surfactant available |
| | from BASF Corporation, Mount Olive, NJ |

### Preparative Example 1

### Preparation of Phosphate Buffer Saline with PLURONIC L64 (PBS-L64 Buffer)

A phosphate buffer saline (PBS) solution was prepared by diluting ten-fold a 10× PBS liquid concentrate (EMD Biosciences, San Diego Calif.). This resulted in a PBS buffer solution with the following salt composition: 10 mM sodium phosphate, 137 mM sodium chloride, 2.7 mM potassium chloride. The PBS buffer solution had a pH of 7.5 at 25° C. PLURONIC L64 surfactant, in the amount of 0.2% (w/v), was added to the PBS buffer solution to provide phosphate buffer saline with PLURONIC L64 (PBS-L64 buffer) with a pH of 7.5 at 25° C.

### Preparative Example 2

### Preparation of Antibody Functionalized Magnetic Beads

All antibody preparations were biotinylated with EZ-Link NHS-PEO4-Biotin (Product Number 21330 from Pierce, Rockford, Ill.) according to the manufacturer's directions. Streptavidin-coated magnetic particles (100 nm FLUIDMAG beads from Chemicell GmbH, Berlin, Germany) were used. All reactions and washes were performed in PBS L-64 buffer unless stated otherwise. Wash steps included three successive washes unless stated otherwise. The washing process consisted of placing a magnet adjacent to the tube to draw the particles to the side of the tube proximal to the magnet, removing the liquid from the tube with the adjacent magnet, and adding an equal volume of fresh buffer to replace the liquid that was removed. The magnet was removed to allow re-suspension and mixing of the particles.

Streptavidin-coated magnetic particles, at a concentration of 2.5 milligram per milliliter (mg/ml) were mixed with biotinylated antibody preparations in 500 μl PBS L-64 buffer. The mass ratio of the antibody to the particles for conjugation was 40 μg antibody/mg of particles. The resulting mixture was incubated at 37° C. for 1 hour (hr). Subsequently, the particles were washed in PBS L-64 buffer to remove unbound antibody. After the final wash the particles were re-suspended to a particle concentration of 2.5 mg/ml.

### Preparative Example 3

### Attachment of Streptavidin or Neutravidin to Polysaccharide or Polystyrene Beads with Carboxyl Functionality on the Surface

A streptavidin stock was prepared by first dissolving 5 mg of ImmunoPure Streptavidin (Pierce, Rockford, Ill.) in 0.5 ml of water (final solution concentration of 10 mg/ml), and subsequently adding 75 μl of the result solution to 425 μl of MES (2-(N-morpholino)ethanesulfonic acid) buffer to obtain a final streptavidin solution concentration of 1.5 mg/ml.

A neutravidin stock was prepared by first dissolving 10 mg of neutravidin (Pierce, Rockford, Ill.) in 1.0 ml of water (final solution concentration of 10 mg/ml), and subsequently adding 75 μl of the resulting solution to 425 μl of MES buffer to obtain a final neutravidin solution concentration of 1.5 mg/ml.

An EDC (1-ethyl-3-(3-dimethylaminopropyl)-carbodiimide) stock solution was prepared just before functionalizing the beads by adding 20 mg EDC to 0.5 ml MES buffer to yield a final EDC solution concentration of 40 mg/ml.

FLUIDMAG ARA beads (100 nm, from Chemicell GmbH, Berlin, Germany) were functionalized with streptavidin or neutravidin using the following procedure:
1. 100 μl of the stock bead solution was washed twice in 1 ml MES buffer, and the supernatant was remove.
2. The beads were re-suspended in 0.2 ml of the EDC solution as prepared above.
3. The suspension was mixed for 10 minutes at room temperature.
4. The beads were washed twice with 1 ml MES buffer.
5. The beads were re-suspended in 0.2 ml MES buffer with the streptavidin or neutravidin stock as prepared above, and the resulting mixture was incubate for 2 hours under agitation at room temperature.
6. The resulting beads were wash three times in PBS L-64 buffer.
7. The beads were re-suspended in 1.2 ml PBS L-64 buffer to provide a final bead solution concentration of 2.5 mg/ml.

DYNAL C1 beads (from Invitrogen Inc., Carlsbad, Calif.) were functionalized with streptavidin or neutravidin using the same procedure outlined above but starting with 300 μl of the bead stock solution rather than 100 μl as above.

### Capture and Detection Procedure for Staphylococcus aureus (S. aureus, ATCCC 25923) and Pseudomonas aeruginosa (P. aeruginosa, ATCCC 10662) Cells

Bacterial suspensions were prepared using overnight cultures grown in tryptic soy broth at 37° C. The cultures were centrifuged to harvest the cells and the cell pellets were re-suspended in sterile phosphate buffered saline to a final concentration of approximately 5×10⁸ cfu/mL. Prior to experimentation, the bacteria were washed in triplicate in PBS L-64 buffer and diluted to approximately 5×10³ to 5×10⁶ cfu/mL. Optionally human serum albumin (HSA) was then added to the solution to achieve a target concentration of protein (typically 300 μg/ml). The desired amount of streptavidin coated particle suspensions (10 mg/mL) (FLUIDMAG beads from Chemicell GmbH, Berlin, Germany) were mixed with the bacteria suspension to a total volume of 500 microliter (μl) in a 2 ml polypropylene vial. The vial was manually rocked for 30 seconds (s) to mix the bacteria and the particles and then agitated on a rocking platform (Reciprocating BARNSTEAD/THERMOLYNE VARIMIX (Dubuque, Iowa)) set at approximately 0.3 cycle per second) for 15 minutes. The particles were drawn to one side of the vial with a magnet for 5 minutes. The supernatant, containing bacterial cells that were not adsorbed by the particles, was removed and diluted 10-fold in sterile PBS L-64 buffer. An optional wash step was then performed by removing the vial from the magnet, adding 500 μl sterile PBS L-64 buffer, and manually agitating the vial for 30 s to re-suspend the particles. The vial was placed adjacent to the magnet for 5 minutes and the wash solution was aspirated and diluted 10-fold in PBS L-64 buffer. The vial was removed from the magnet, 500 μl buffer was added, and the vial was manually agitated for 30 s to re-suspend the particles.

The number of viable bacteria in each of the respective solutions (supernatant, wash and re-suspended particles) was determined by plating serial dilutions of each suspension on PETRIFILM Aerobic Count Plates (3M Company, St. Paul, Minn.).

### Example 1

### S. aureus (ATCCC 25923) Capture

FLUIDMAG particles (100nm, from Chemicell GmbH, Berlin, Germany) with a polysaccharide core and functionalized with streptavidin were used to non-specifically capture S. aureus 25923 using the above Capture and Detection Procedure. 10 μl of particles were combined with 490 μl of the bacteria at approximately 5×10³ cfu/ml. A wash step was included in the experiment.

The particles demonstrated high % capture of the bacteria. When the same particles were coated with an antibody (as prepared by Preparative Example 2) that was not specific to any antigens present on the cell surface, and the same experiment repeated, the capture was poor. Thus the presence of the antibody significantly reduced the non-specific bacteria capture. The results are shown in Table 1.

TABLE 1
S. aureus Capture with 100 nm Beads with a Polysaccharide
Core and Functionalized with Streptavidin

| Particles | % Capture |
|---|---|
| streptavidin on polysaccharide | 96.7 |
| PBP2A antibody-biotin attached to | 11.9 |
| streptavidin on polysaccharide | |

For PBP2A antibody, see Assignee's co-pending U.S. patent application Ser. No. 60/867,089.

### Example 2

### Comparison Between Streptavidin Functionalized Beads and Carboxyl Functionalized Beads Without Streptavidin

FLUIDMAG magnetic particles with a polysaccharide core and functionalized with streptavidin (100 nm, Chemicell GmbH) were used to non-specifically capture bacteria according to the above Capture and Detection Procedure. In addition, FLUIDMAG ARA magnetic particles with a polysaccharide core and surface carboxyl surface groups (100 nm, Chemicell GmbH) were also tested. The capture was performed for both P. aeruginosa (ATCCC 10662) (Table 2) and S. aureus (ATCCC 25923) (Table 3). Capture was tested with and without the presence of human serum albumin 300 μg/ml. The particles (30 μl) were combined with 470 μl of the bacteria (particle concentration of 0.6 mg/ml) at approximately 5×10⁶ cfu/ml for all of the experiments. The results are shown in Tables 2 and 3.

TABLE 2
P. aeruginosa (10662) Capture by Streptavidin on Polysaccharide
Compared with Carboxyl on Polysaccharide Without Streptavidin

| Particle surface | Serum albumin (μg/ml) | Rep 1 | Rep 2 |
|---|---|---|---|
| streptavidin on | 0 | 47.51 | 47.70 |
| polysaccharide | | | |
| streptavidin on | 300 | 43.44 | 33.66 |
| polysaccharide | | | |
| carboxyl on | 0 | 26.21 | 24.11 |
| polysaccharide | | | |
| carboxyl on | 300 | 9.97 | 9.42 |
| polysaccharide | | | |

As shown in Table 2, the streptavidin beads showed capture of P. aeruginosa with a slightly lower capture in the presence of serum albumin. However, the carboxyl beads showed significantly lower capture, especially in the presence of serum albumin. Even though both particles had the same polysaccharide core chemistry, the particles with streptavidin demonstrated much better capture as compared to the particles surface functionalized with carboxyl groups but without streptavidin.

TABLE 3
S. aureus (25923) Capture by Streptavidin on Polysaccharide
Compared with Carboxyl on Polysaccharide Without Streptavidin

| Particle surface | Serum albumin (μg/ml) | Rep 1 | Rep 2 |
|---|---|---|---|
| streptavidin on | 0 | 99.78 | 99.91 |
| polysaccharide | | | |
| streptavidin on | 300 | 99.58 | 95.95 |
| polysaccharide | | | |
| carboxyl on | 0 | 4.21 | 9.15 |
| polysaccharide | | | |
| carboxyl on | 300 | 6.20 | 3.30 |
| polysaccharide | | | |

As shown in Table 3, the streptavidin/polysaccharide beads showed excellent capture of S. aureus both in the presence and absence of serum albumin. However, the carboxyl beads without streptavidin showed poor capture in both cases. Even though both particles had the same polysaccharide core, the particles with streptavidin on the surface performed significantly better.

### Example 3

### Capture by Streptavidin on Polysaccharide in the Presence of Red Blood Cells and Proteins

FLUIDMAG magnetic particles (100 nm, Chemicell GmbH) with a polysaccharide core and functionalized with streptavidin were used to non-specifically capture bacteria according to the above Capture and Detection Procedure. Capture was performed for both P. aeruginosa (ATCCC 10662) (Table 4) and S. aureus (ATCCC 25923) (Table 5). Capture was carried out in the presence of blood in the sample at varying concentrations (1:1000, 1:10000 and 1:100000 dilutions of whole blood). A control sample with no blood was also tested. The particles (30 μl) were combined with 470 μl of the bacteria (particle concentration of 0.6 mg/ml) at approximately 5×10⁶ cfu/ml for all of the experiments. The results are shown in Tables 4 and 5.

TABLE 4
P. aeruginosa (10662) Capture by Streptavidin on Polysaccharide
in the Presence and Absence of Blood Cells

| Blood Dilution | Rep 1 | Rep 2 | Rep 3 |
|---|---|---|---|
| 1:1000 | 69.59 | 72.43 | 72.32 |
| 1:10000 | 88.41 | 92.96 | 91.84 |
| 1:100000 | 90.91 | 96.49 | 96.50 |
| Control (no blood) | 98.71 | 98.42 | 99.95 |

Excellent capture of P. aeruginosa was seen in the control sample. Although capture decreased with increasing levels of blood, the capture levels were satisfactory even at the highest levels of blood tested (1:1000 dilution).

TABLE 5
S. aureus (25923) Capture by Streptavidin on Polysaccharide
in the Presence and Absence of Blood Cells

| Blood Dilution | Rep 1 | Rep 2 | Rep 3 |
|---|---|---|---|
| 1:1000 | 73.9 | 81.4 | 82.1 |
| 1:10000 | 60.4 | 90.7 | 91.6 |
| 1:100000 | 98.0 | 97.3 | 97.9 |
| Control (no blood) | 99.2 | 99.5 | 99.9 |

For S. aureus, excellent capture was seen in the control sample. Although capture decreased with increasing levels of blood, the capture levels were satisfactory even at the highest levels of blood tested (1:1000 dilution).

### Example 4

### S. aureus (ATCCC 25923) Capture by Streptavidin on Polysaccharide Beads Compared with Capture by Streptavidin on Polystyrene-Carboxyl Beads

Streptavidin was attached to polystyrene-carboxyl beads (DYNAL C1) and polysaccharide-carboxyl beads (FLUIDMAG ARA, Chemicell GmbH, Berlin, Germany) as in Preparative Example 3, and S. aureus (ATCCC 25923) was captured according to the above Capture and Detection Procedure (32 μl of the stock bead solution as prepared in Preparative Example 3 was added to 468 μl of the bacteria stock sample to obtain a final bead solution concentration of 0.16 mg/ml). In addition, capture experiments were performed with the unmodified polystyrene-carboxyl (DYNAL C1) and polysaccharide-carboxyl (FLUIDMAG ARA) beads. The results are shown in Table 6.

TABLE 6
S. aureus (ATCCC 25923) Capture by Streptavidin on
Polysaccharide, Polysaccharide-Carboxyl, Streptavidin
on Polystyrene-Carboxyl, and Polystyrene-Carboxyl Beads
(Stock bacterial concentration was 44000 cfu/ml.)

| | Bacteria count | Bacteria count | Avg. Bacteria count |
|---|---|---|---|
| Bead type | on beads | on beads | on beads |
| Polystyrene | 12000 | 11500 | 11750 |
| carboxyl | | | |
| Polysaccharide | 950 | 1000 | 975 |
| carboxyl | | | |
| Polystyrene | 2300 | 2550 | 2425 |
| streptavidin | | | |
| Polysaccharide | 29500 | 31250 | 30375 |
| streptavidin | | | |

Referring to Table 6, the capture efficiency of the streptavidin functionalized polystyrene beads was about 5 times lower than the unmodified polystyrene beads. On the other hand, for the polysaccharide beads modified with streptavidin, the capture efficiency was approximately ten times better than either the unmodified polysaccharide beads or the polystyrene beads functionalized with streptavidin.

### Example 5

### P. aeruginosa (ATCCC 35032) Capture by Streptavidin on Polysaccharide Beads Compared with Capture by Streptavidin on Polystyrene-Carboxyl Beads

Using P. aeruginosa (ATCCC 35032) instead of S. aureus, Example 4 was essentially repeated, and the results are summarized below in Table 7. Overall, the polysaccharide beads modified with streptavidin provided the best non-specific bacteria capture performance.

TABLE 7
P. aeruginosa Capture by Streptavidin on Polysaccharide,
Polysaccharide-Carboxyl, Streptavidin on Polystyrene-
Carboxyl, and Polystyrene-Carboxyl Beads (Stock bacterial
concentration was 12000 cfu/ml.)

| | Bacteria count | Bacteria count | Avg. Bacteria count |
|---|---|---|---|
| Bead type | on beads | on beads | on beads |
| Polystyrene | 1300 | 800 | 1050 |
| carboxyl | | | |
| Polysaccharide | 4450 | 5100 | 4775 |
| carboxyl | | | |
| Polystyrene | 3500 | 3750 | 3625 |
| streptavidin | | | |
| Polysaccharide | 5250 | 5150 | 5200 |
| streptavidin | | | |

### Example 6

### S. aureus (ATCCC 25923) Capture by Neutravidin on Polysaccharide Beads Compared with Capture by Neutravidin on Polystyrene-Carboxyl Beads

Neutravidin was attached to polystyrene-carboxyl (DYNAL C1) and polysaccharide-carboxyl (FLUIDMAG ARA, Chemicell GmbH) beads as in Preparative Example 3, and S. aureus (ATCCC 25923) was captured according to the above Capture and Detection Procedure (32 μl of the stock bead solution as prepared in Preparative Example 3 was added to 468 μl of the bacteria stock sample to obtain a final bead solution concentration of 0.16 mg/ml). The results are shown in Table 8.

TABLE 8
S. aureus Capture by Neutravidin on Polysaccharide
Beads Compared with Neutravidin on Polystyrene Beads
(Stock bacterial concentration was 88000 cfu/ml.)

| | Bacteria count | Bacteria count | Avg. Bacteria count |
|---|---|---|---|
| Bead type | on beads | on beads | on beads |
| Polystyrene | 700 | 600 | 650 |
| neutravidin | | | |
| Polysaccharide | 47000 | 57000 | 52000 |
| neutravidin | | | |

Referring to Table 8, the polysaccharide beads modified with neutravidin showed capture efficiencies ten times better than the polystyrene beads functionalized with neutravidin.

### Example 7

### P. aeruginosa (ATCCC 35032) Capture by Neutravidin on Polysaccharide Beads Compared with Capture by Neutravidin on Polystyrene-Carboxyl Beads

Using P. aeruginosa (ATCCC 35032) instead of S. aureus, Example 6 was essentially repeated, and the results are summarized below in Table 9. The polysaccharide beads with neutravidin provided significantly greater non-specific bacterial capture than the polystyrene beads with neutravidin.

TABLE 9
P. aeruginosa Capture by Neutravidin on Polysaccharide
Beads Compared with Neutravidin on Polystyrene Beads
(Stock bacterial concentration was 18000 cfu/ml.)

| | Bacteria count | Bacteria count | Avg. Bacteria count |
|---|---|---|---|
| Bead type | on beads | on beads | on beads |
| Polystyrene | 1600 | 650 | 1125 |
| neutravidin | | | |
| Polysaccharide | 4850 | 3300 | 4075 |
| neutravidin | | | |

### Example 8

### Non-specific Capture of Various Bacteria Using Polysaccharide Beads Functionalized with Streptavidin

Streptavidin was attached to polysaccharide-carboxyl beads (FLUIDMAG ARA, Chemicell GmbH) as in Preparative Example 3, and various bacteria strains (from clinical isolates) were captured according to the above Capture and Detection Procedure (32 μl of the stock bead solution as prepared in Preparative Example 3 was added to 468 μl of the bacteria stock sample to obtain a final bead solution concentration of 0.16 mg/ml). The results are summarized below in Table 10.

TABLE 10
Non-specific Capture of Various Bacteria Using Polysaccharide
Beads Functionalized with Streptavidin

| | Bacteria count | Bacteria count | Initial stock |
|---|---|---|---|
| Bacteria | on beads | on beads | concentration |
| S. aureus ATCCC | 14000 | 16000 | 25000 |
| 25923 | | | |
| S. aureus 050 | 9000 | 8600 | 32000 |
| S. aureus 27A | 5900 | 7600 | 33000 |
| S. epidermidis 31A | 23000 | 27000 | 28000 |
| E. faecalis 32A | 3600 | 2400 | 19000 |
| P. aeruginosa 4A | 11000 | 10000 | 28000 |
| P. aeruginosa 26A | 2100 | 1700 | 20000 |

The results in Table 10 show that the polysaccharide-streptavidin beads can be used to non-specifically capture a variety of microorganisms.

### Example 9

### Non-specific Capture of Streptococcus Bacteria Strains Using Streptavidin Functionalized Polysaccharide Beads

FLUIDMAG magnetic particles (100 nm, Chemicell GmbH) with a polysaccharide core and functionalized with streptavidin were used to non-specifically capture Strep agalatiae 39B and Strep dysgalatiae 1E (clinical isolates). The above Capture and Detection Procedure was followed, using a bead solution concentration of 0.6 mg/ml. The results (shown in Table 11) demonstrate efficient capture of both Strep agalatiae and Strep dysgalatiae.

TABLE 11
Non-specific Capture of Streptococcus Bacteria
Strains Using FLUIDMAG Beads (Chemicell GmbH)

| | Bacteria count | Bacteria count | Stock |
|---|---|---|---|
| Bacteria | on beads | on beads | concentration |
| Strep agalatiae 39B | 3300 | 3400 | 5600 |
| Strep dysgalatiae 1E | 7600 | 8800 | 9400 |

### Example 10

### Effect of Saponin on Non-specific Capture of Bacteria Using Polysaccharide Beads Functionalized with Streptavidin

E. coli, Salmonella, and Group B Streptococcus solutions were prepared by growing these bacteria over night on blood agar plates. Bacteria suspensions were then prepared in PBS L-64 buffer by MacFarland turbidity standards to a concentration of approximately 1×10⁸ cfu/mL. The suspensions were diluted in PBS L-64 buffer to a working concentration of approximately 1×10⁶ cfu/mL.

Capture experiments were performed by adding 32 μl of streptavidin functionalized polysaccharide-carboxyl beads (made using FLUIDMAG ARA beads in Preparative Example 3) (2.5 mg/mL) to 234 μl of the bacteria sample. A 2% stock solution of saponin (Product #47036, Sigma-Aldrich) in PBS buffer was prepared. For capture experiments in the presence of saponin, 234 μl of the stock saponin solution was added to the bead-bacteria mixture, resulting in a 0.9% concentration of saponin during capture. For capture experiments without saponin, 234 μl of PBS L-64 buffer was added to the bead-bacteria mixture. The bead concentration was 0.16 mg/ml during the capture experiments, which were carried out from this point forward as described in the above Capture and Detection Procedure. The results shown in Table 12 indicate that the presence of saponin increased bacteria capture for all bacteria tested.

TABLE 12
The Effect of Saponin on Non-specific Capture of E. coli,
Salmonella, and Group B Streptococcus Using
Streptavidin Functonalized Polysaccharide Beads

| | Bacteria count on beads | | | | Stock |
|---|---|---|---|---|---|
| | no saponin | no saponin | w saponin | w saponin | Concen- |
| Bacteria | Rep 1 | Rep 2 | Rep 1 | Rep 2 | tration |
| E. coli | 1000 | 3000 | 29000 | 37000 | 92000 |
| E. coli | 6000 | 7000 | 59000 | 61000 | 210000 |
| Salmonella | 890000 | 780000 | 1100000 | 1300000 | 1600000 |
| Group B | 44000 | 66000 | 190000 | 195000 | 900000 |
| Strep | | | | | |

### Examples 11-22 and Comparative Examples 1-4

### Capture of Bacteria and ATP Detection

FLUIDMAG ARA beads (100 nm, from Chemicell GmbH, Berlin, Germany) with a polysaccharide core and functionalized with streptavidin or neutravidin (prepared as in Preparative Example 3) were used to non-specifically capture S. aureus (ATCC 25923) and E. coli (ATCC 25922) using the above Capture and Detection Procedure. For each example, 10 μl of the bead suspension was combined with 490 μl of the bacteria suspensions at two concentrations: ˜1×10⁶ cfu/ml and ˜1×10⁵ cfu/ml, using polypropylene microcentrifuge tubes (available from VWR Scientific, West Chester, Pa.). Samples with 0 cfu/ml and positive control samples (bacteria at either 1×10⁶ cfu/ml or 1×10⁵ cfu/ml without any capture beads) were also prepared using the above Capture and Detection Procedure.

The beads were then separated and concentrated by placing the microcentrifuge tube in a DYNAL magnetic fixture (available from Invitrogen, Inc. Carlsbad, Calif.) for at least 5 minutes. The supernatant was discarded by micropipetting without disrupting the agglomerated beads.

The beads were then washed by adding 0.5 mL PBS-L64 buffer to the tube and agitating using a rocking motion for 5 minutes. The beads were then again separated and concentrated by placing the microcentrifuge tube in the DYNAL magnetic fixture for at least 5 minutes. The wash solution was discarded by micropipetting without disrupting the agglomerated beads. This wash step was repeated a second time.

100 μl of DEPC (diethyl pyrocarbonate, available from Aldrich Chemicals, Milwaukee Wis.)) treated water and 100 μl of Extractant XM (available from Biotrace International BioProducts Inc., Bothell, Wash.) were added to the beads. The vial was removed from the magnetic fixture, and manually agitated to resuspend the particles. Vortexing was also used to make sure the particles were suspended without any visible aggregation. The suspended particles were incubated with the DEPC treated water and Extractant XM for a minimum of 60 seconds.

The beads were then again separated and concentrated by placing the microcentrifuge tube in the DYNAL magnetic fixture for at least 5 minutes. The supernatant was then micropipetted to the bottom chamber of a Biotrace AQUA-TRACE test device (available from Biotrace International BioProducts Inc., Bothell, Wash.) after removal of the swab and the foil sealed chamber containing lysing agents in pelletized form. The bottom chamber of the Biotrace device contained all the necessary dry reagents to determine the presence of ATP via luciferase bioluminescence. The sample was vortexed for 10 seconds and placed in a Biotrace luminometer (UNI-LITE NG, (available from Biotrace International BioProducts Inc., Bothell, Wash.)) within thirty seconds after adding the supernatant to the bottom chamber of a Biotrace AQUA-TRACE test device. The bioluminescent response from each sample is reported in Table 13 in Relative Light Units (RLUs). The average RLUs from three replicates is reported in Table 13. Also shown in Table 13 are the 1 sigma standard deviations on the reported RLUs values.

TABLE 13
Detection of captured S. aureus and E. coli with ATP detection

| | Bead Type | S. aureus | E. coli | Relative | |
|---|---|---|---|---|---|
| | (Biotin | Concen- | Concen- | Light | 1 sigma |
| Exam- | binding | tration | tration | Units | standard |
| ple | protein) | (cfu/mL) | (cfu/mL) | (RLU) | deviation |
| 11 | Streptavidin | 0 | 0 | 9 | 1 |
| 12 | Streptavidin | 100,000 | 0 | 265 | 6 |
| 13 | Streptavidin | 1,000,000 | 0 | 1165 | 67 |
| 14 | Neutravidin | 0 | 0 | 10 | 0.5 |
| 15 | Neutravidin | 100,000 | 0 | 210 | 5 |
| 16 | Neutravidin | 1,000,000 | 0 | 1123 | 70 |
| 17 | Streptavidin | 0 | 0 | 4 | 1 |
| 18 | Streptavidin | 0 | 100,000 | 12 | 1 |
| 19 | Streptavidin | 0 | 1,000,000 | 95 | 11 |
| 20 | Neutravidin | 0 | 0 | 10 | 0.5 |
| 21 | Neutravidin | 0 | 100,000 | 20 | 2 |
| 22 | Neutravidin | 0 | 1,000,000 | 100 | 7 |
| C1 | None | 100,000 | 0 | 480 | 10 |
| C2 | None | 1,000,000 | 0 | 4913 | 59 |
| C3 | None | 0 | 100,000 | 503 | 9 |
| C4 | None | 0 | 1,000,000 | 5105 | 62 |

C1, C2, C3, and C4 are Comparative Examples 1, 2, 3, and 4.

S. aureus was detected at both concentrations tested, whereas E. coli was detected only at the highest concentration tested. When comparing the positive control signal intensities (C1-C4) to that of samples captured using the paramagnetic beads, lower intensities found for the captured samples originated from less than 100 percent capture efficiency of the sample preparation but also the loss of sample ATP due to non-specific binding of the extracted ATP on the beads during the assay.

### Examples 23-27

### Capture and ATP Detection of S. aureus and E. coli in the Presence of Lysed Blood Cells

The assay to detect bacteria was repeated exactly as described in Examples 11-22, except for an initial sample composition which contained: 234 μl of a 1:10,000 diluted (using PBS buffer) and lysed (using 0.9% Saponin Product #47036, Sigma-Aldrich) human whole blood specimen, along with 234 μl of PBS buffer containing bacteria at either 0, 1×10⁵, or 1×10⁶ cfu/ml, and 32 μl of the FLUIDMAG particles (100 nm, from Chemicell GmbH, Berlin, Germany) with a polysaccharide core and functionalized with streptavidin. The bioluminescent response from each sample is reported in Table 14 in Relative Light Units (RLUs). The average RLUs from three replicates is reported in Table 14. Also shown in Table 14 are the 1 sigma standard deviations on the reported RLUs values. The data shown in Table 14 demonstrates that detection of both bacteria was observed at both concentrations tested.

TABLE 14
ATP detection if S. aureus and E. coli
in the presence of lysed blood cells

| | Bead Type | S. aureus | E. coli | Relative | |
|---|---|---|---|---|---|
| | (Biotin | Concen- | Concen- | Light | 1 sigma |
| Exam- | binding | tration | tration | Units | standard |
| ple | protein) | (cfu/mL) | (cfu/mL) | (RLU) | deviation |
| 23 | Streptavidin | 0 | 0 | 27 | 5 |
| 24 | Streptavidin | 100,000 | 0 | 271 | 10 |
| 25 | Streptavidin | 1,000,000 | 0 | 1296 | 57 |
| 26 | Streptavidin | 0 | 100,000 | 254 | 8 |
| 27 | Streptavidin | 0 | 1,000,000 | 1473 | 51 |

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A composition comprising:
a solid support which has a surface comprising a combination of a carbohydrate and a biotin-binding protein, wherein the protein is covalently bonded to the carbohydrate, and wherein the protein is linked to the solid support via the carbohydrate; and
a plurality of bacterial cells non-specifically bound to the combination of the carbohydrate and the protein.

2. A composition comprising:
a solid support which has a surface comprising a combination of a carbohydrate and a biotin-binding protein, wherein the protein is covalently bonded to the carbohydrate, and wherein the protein is linked to the solid support via the carbohydrate; and
an amphiphilic glycoside of a steroid or triterpene.

3. The composition of claim 2, further comprising a plurality of bacterial cells non-specifically bound to the combination of the carbohydrate and the protein.

4. The composition of claim 1, wherein the plurality of bacterial cells includes two or more different types of bacteria.

5. (canceled)

6. The composition of claim 2, wherein the amphiphilic glycoside of a steroid or triterpene is saponin.

7. The composition of claim 1, wherein the biotin-binding protein is selected from the group consisting of avidin, streptavidin, neutravidin, and selectively nitrated avidin; wherein the carbohydrate includes at least one carboxy group, and wherein the biotin-binding protein is covalently bonded to the carbohydrate through a linking group, the linking group being the reaction product of the protein and the at least one carboxy group of the carbohydrate.

8. (canceled)

9. The composition of claim 2, wherein the biotin-binding protein is selected from the group consisting of avidin, streptavidin, neutravidin, and selectively nitrated avidin; wherein the carbohydrate includes at least one carboxy group, and wherein the biotin-binding protein is covalently bonded to the carbohydrate through a linking group, the linking group being the reaction product of the protein and the at least one carboxy group of the carbohydrate.

10-12. (canceled)

13. A method for isolating bacterial cells comprising:
providing a solid support which has a surface comprising a combination of a carbohydrate and a biotin-binding protein, wherein the protein is covalently bonded to the carbohydrate, and wherein the protein is linked to the solid support via the carbohydrate;
providing a sample suspected of having a plurality of bacterial cells;
contacting the solid support which has the surface comprising the combination of the carbohydrate and the biotin-binding protein with the sample; wherein at least a portion of the plurality of bacterial cells from the sample become non-specifically bound to the surface of the solid support; and
separating the solid support from the remainder of the sample after the at least a portion of the plurality of bacterial cells from the sample become non-specifically bound to the surface of the solid support.

14-15. (canceled)

16. The method of claim 13, wherein contacting the solid support with the sample is carried out in the presence of an amphiphilic glycoside of a steroid or triterpene.

17. The method of claim 16, wherein the amphiphilic glycoside of a steroid or triterpene is saponin.

18. The method of claim 13, wherein the biotin-binding protein is selected from the group consisting of avidin, streptavidin, neutravidin, and selectively nitrated avidin.

19. (canceled)

20. The method of claim 13, wherein the carbohydrate includes at least one carboxy group, and wherein the biotin-binding protein is covalently bonded to the carbohydrate through a linking group, the linking group being the reaction product of the protein and the at least one carboxy group of the carbohydrate.

21-23. (canceled)

24. The method of claim 13, wherein the plurality of bacterial cells includes two or more different types of bacteria.

25. (canceled)

26. A device for detecting bacterial cells comprising:
a composition comprising:
a solid support which has a surface comprising a combination of a carbohydrate and a biotin-binding protein, wherein the protein is covalently bonded to the carbohydrate, and wherein the protein is linked to the solid support via the carbohydrate; and
a plurality of bacterial cells non-specifically bound to the combination of the carbohydrate and the protein; and
a means for detecting the bacterial cells.

27. A device for detecting bacterial cells comprising:
a composition comprising:
a solid support which has a surface comprising a combination of a carbohydrate and a biotin-binding protein, wherein the protein is covalently bonded to the carbohydrate, and wherein the protein is linked to the solid support via the carbohydrate; and
an amphiphilic glycoside of a steroid or triterpene; and
a means for detecting the bacterial cells.

28-32. (canceled)

33. The device of claim 26, wherein the biotin-binding protein is selected from the group consisting of avidin, streptavidin, neutravidin, and selectively nitrated avidin; wherein the carbohydrate includes at least one carboxy group, and wherein the biotin-binding protein is covalently bonded to the carbohydrate through a linking group, the linking group being the reaction product of the protein and the at least one carboxy group of the carbohydrate.

34. (canceled)

35. The device of claim 27, wherein the biotin-binding protein is selected from the group consisting of avidin, streptavidin, neutravidin, and selectively nitrated avidin; wherein the carbohydrate includes at least one carboxy group, and wherein the biotin-binding protein is covalently bonded to the carbohydrate through a linking group, the linking group being the reaction product of the protein and the at least one carboxy group of the carbohydrate.

36-38. (canceled)

39. A kit comprising:
a solid support which has a surface comprising a combination of a carbohydrate and a biotin-binding protein, wherein the protein is covalently bonded to the carbohydrate, and wherein the protein is linked to the solid support via the carbohydrate; and
an amphiphilic glycoside of a steroid or triterpene.

40-42. (canceled)

43. The kit of claim 39, wherein the biotin-binding protein is selected from the group consisting of avidin, streptavidin, neutravidin, and selectively nitrated avidin; wherein the carbohydrate includes at least one carboxy group, and wherein the protein is covalently bonded to the carbohydrate through a linking group, the linking group being the reaction product of the protein and the at least one carboxy group of the carbohydrate.

44-48. (canceled)

49. The composition of claim 3, wherein the plurality of bacterial cells includes two or more different types of bacteria.
